(19) United States
(12) Patent Application Publication
(10) Pub. No.: US 2024/0408039 A1
(43) Pub. Date: Dec. 12, 2024

(54) NOREPINEPHRINE LIQUID FORMULATIONS

(71) Applicant: Fresenius Kabi USA, LLC, Lake Zurich, IL (US)

(72) Inventors: Arunya USAYAPANT, Lake Zurich, IL (US); Chaoju XIAO, Lake Zurich, IL (US)

(73) Assignee: Fresenius Kabi USA, LLC, Lake Zurich, IL (US)

(21) Appl. No.: 18/715,636

(22) PCT Filed: Nov. 28, 2022
(86) PCT No.: PCT/US2022/080492

§ 371 Date: May 31, 2024

Related U.S. Application Data
(60) Provisional application No. 63/286,029, filed on Dec. 4, 2021.

(51) Int. Cl. A61K 31/137; A61J 1/10; A61J 1/14; A61K 47/18

(52) U.S. Cl. CPC A61K 31/137; A61J 1/10; A61J 1/1468; A61K 47/183

(57) ABSTRACT

The invention provides a liquid formulation comprising norepinephrine or a pharmaceutically acceptable salt thereof. The formulation invention includes an antioxidant, an isotonicity agent, and has a pH of about 3.0-3.8. The liquid formulation according to the invention is stable and ready-to-administer.

### BACKGROUND OF THE INVENTION

Norepinephrine is a phenylethamine and catecholamine that functions in the body as a neurotransmitter and a hormone. In the brain, norepinephrine increases arousal, enhances memory, and focuses attention while also increasing restlessness and anxiety. In the rest of the body, norepinephrine increases heart rate and blood pressure, triggers glucose release from energy stores, and increases blood flow to skeletal muscle.

Norepinephrine also is referred to as noradrenalin, noradrenaline, (R)-(−)-norepinephrine, 4-[(1R)-2-amino-1-hydroxyethyl]benzene-1,2-diol, and 1-1-(3,4-dihydroxyphenyl)-2-aminoethanol. Norepinephrine has a molecular weight of approximately 169.18 and the following chemical structure:

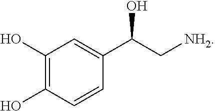 [SMILES: NC[C@H](O)c1ccc(O)c(O)c1]

In medicine, injections of norepinephrine, or a pharmaceutically acceptable salt thereof, are used for the treatment of critically low blood pressure. LEVOPHED™ (norepinephrine bitartrate) injection has been available in the United States for several decades as a sterile aqueous solution containing 1 mg/mL of norepinephrine base (equivalent to 1.89 mg of norepinephrine bitartrate, anhydrous basis), sodium chloride for isotonicity, sodium metabisulfite as an antioxidant, and having a pH of 3.0 to 4.5. The prescribing information for LEVOPHED™ (norepinephrine bitartrate) injection states that the product must be diluted before use, as follows. Add the content of one LEVOPHED vial or ampule (4 mg in 4 mL) to 1,000 mL of 5% Dextrose Injection, USP or Sodium Chloride Injection solutions that contain 5% dextrose to produce a 4 mcg per mL dilution. Dextrose reduces loss of potency due to oxidation. Administration in saline solution alone is not recommended. Use higher concentration solutions in patients requiring fluid restriction. Prior to use, store the diluted LEVOPHED solution for up to 24 hours at room temperature [20° C. to 25° C. (68° F. to 77° F.)] and protect from light. Pharmaceutical products containing 1 mg/mL of norepinephrine are available from several generic pharmaceutical manufacturers.

Other injectable formulations of norepinephrine have been described. U.S. Pat. Nos. 9,877,935 and 11,166,923 disclose a ready-to-administer parenteral dosage form of norepinephrine which comprises an aqueous solution of norepinephrine, having an anti-oxidant which is not a sulfite anti-oxidant selected from butylated hydroxyl anisole, ascorbic acid, propyl gallate, vitamin E or alpha-tocopherol, and a pH of about 3.0 to about 4.5.

U.S. Pat. Nos. 10,159,657, 10,226,436, 10,420,735, 10,471,026, 10,568,850, and 10,646,458 and U.S. Patent Application Publication No. 2020/0230079 disclose ready-to-inject norepinephrine compositions that are substantially antioxidant free, contain a chelating agent such as a dicarboxylic acid, a tricarboxylic acid, and an aminopolycarboxylic acid, and have a pH between 3.7-5.0.

U.S. Pat. No. 10,251,848 discloses an injectable noradrenaline solution comprising noradrenaline, a solvent, an excipient, and hydrochloric acid, wherein the amount of noradrenaline is from 0.04 to 0.2 mg/ml, the solvent is degassed or deaerated water, the excipient is NaCl, and the pH of the solution is in the range of from 3.3 to 3.6, and wherein the solution is free of preservatives and anti-oxidizing agents.

U.S. Pat. No. 10,888,534 discloses a sterile, antioxidant-free aqueous norepinephrine solution packaged in a flexible plastic container in a sealed over-wrap pouch containing an oxygen absorber consisting essentially of between about 0.010 and 0.2 mg/ml of norepinephrine concentration as free base, a tonicity adjusting agent to provide an osmolality of from 260 and 320 mOsm/kg, and sufficient acid and optionally a base to provide a pH of from about 3.6 to 3.8, with the remainder water, and wherein dissolved oxygen in the solution has not been removed before filling the flexible plastic container with the solution.

U.S. Patent Application Publication No. 2021/0038539 discloses a ready to use aqueous dosage form of norepinephrine comprising an aqueous solution of norepinephrine or its pharmaceutically acceptable salt, one or more sulfite antioxidants, and an ion chelator provided in an infusion container, wherein the solution can be terminally sterilized by autoclaving.

U.S. Patent Application Publication No. 2021/0275470 discloses a packaged, sealed container system for a ready-to-use norepinephrine formulation. The system includes a primary container comprising therein an antioxidant-free formulation comprising 0.01 mg/mL to less than 0.04 mg/mL norepinephrine and an aqueous tonicity adjusting agent having a pH 3.4-4.0 that is disposed within a secondary container comprising first and second flexible sheet layers sealed along a common peripheral edge. The system further includes an oxygen scavenger disposed between and enclosed by the first and second flexible sheet layers of the secondary container and being in fluid communication with the contents of the primary container.

A pharmaceutical product containing 16 mcg/mL or 32 mcg/mL norepinephrine in 5% dextrose was approved for use in the United States in January 2021 under New Drug Application (NDA) 214313. The prescribing information states that the product is supplied as a sterile aqueous solution administered by intravenous infusion. Each mL contains the equivalent of 16 or 32 micrograms of norepinephrine base supplied as 31.90 and 63.80 micrograms per mL of norepinephrine bitartrate monohydrate. It contains dextrose monohydrate (50 mg/mL) and may contain hydrochloric acid and/or sodium hydroxide for pH adjustment. It has a pH of 3.5-3.9. The air in the containers has been displaced by nitrogen gas. The prescribing information also states that no further dilution prior to infusion is required. The approval letter for NDA 214313 states that an expiration period of 7 months is granted for the product when stored at 20° C.-25° C. (68° F.-77° F.) in the commercial packaging.

There remains a need in the art for improved ready-to-administer, injectable formulations of norepinephrine which are storage stable, preferably for longer durations at room temperature.

### BRIEF SUMMARY OF THE INVENTION

The invention provides a ready-to-administer, liquid formulation comprising a pharmaceutically effective amount of norepinephrine or a pharmaceutically acceptable salt thereof, an antioxidant, and a tonicity agent, wherein the formulation is stable for at least about 9 months at room temperature.

The invention also provides a method for making a ready-to-administer, liquid norepinephrine formulation that is stable for at least about 9 months at room temperature. The method comprises (1) dissolving a tonicity agent and an antioxidant in water to form a first solution, (2) adjusting the first solution to a pH of from about 3.0 to about 3.8 to form a second solution, (3) dissolving a pharmaceutically effective amount of norepinephrine or a pharmaceutically acceptable salt thereof in the second solution to form a norepinephrine solution, and (4) sterilizing the norepinephrine solution to provide the ready-to-administer, liquid norepinephrine formulation.

### DETAILED DESCRIPTION OF THE INVENTION

The invention provides a liquid formulation comprising a pharmaceutically effective amount of norepinephrine or a pharmaceutically acceptable salt thereof, an antioxidant, and a tonicity agent. The liquid formulation according to the invention exhibits improved stability over comparable conventional formulations, and is ready-to-administer.

As used herein, a “ready-to-administer” formulation refers to a sterile, injectable formulation that need not be reconstituted from a solid or diluted from a concentrated solution by a healthcare provider prior to use. Rather, in the context of norepinephrine formulations of the invention, a ready-to-administer formulation is supplied by a pharmaceutical manufacturer as a liquid having a pharmaceutically effective amount of norepinephrine dissolved therein and contained within a suitable container (e.g., a bag or bottle) along with instructions indicating that no further dilution prior to injection or infusion is required.

The formulation according to the present invention is stable. As used herein, the terms “stable” and “stability” encompass any characteristic of the formulation which may change or be affected by storage conditions including, without limitation, potency, total impurities, norepinephrine degradation products, specific optical rotation, optical purity, appearance, viscosity, sterility, particulates (visible and subvisible), color, and/or clarity. The storage conditions which may affect stability may include, for example, duration, temperature, humidity, and/or light exposure.

For example, a stable norepinephrine formulation may refer to a formulation that contains at least about 90%, e.g., least about 95%, at least about 96%, or at least about 98%, of the labeled concentration of norepinephrine or pharmaceutically acceptable salt thereof after storage under room temperature (e.g., 25° C.±2° C./60% relative humidity (RH)±5% RH) and/or accelerated (e.g., at 40° C.±2° C./75% RH±5% RH) conditions. A stable norepinephrine formulation also may refer to a formulation that contains less than about 110%, e.g., less than about 105%, less than about 103%, or less than about 102%, of the of the labeled concentration of norepinephrine or pharmaceutically acceptable salt thereof after storage under room temperature and/or accelerated conditions. A stable norepinephrine formulation additionally may refer to a formulation that contains from about 95% to about 105%, e.g., from about 97% to about 103%, from about 98% to about 102%, or from about 99% to about 101%, of the labeled concentration of norepinephrine or pharmaceutically acceptable salt thereof after storage under room temperature and/or accelerated conditions.

A a stable norepinephrine formulation also may refer to a formulation that contains less than about 10% (area percent), e.g., less than about 8%, less than about 6%, less than about 5%, less than about 4%, less than about 3%, less than about 2%, or less than about 1%, of total norepinephrine-related impurities present in the formulation after storage under room temperature and/or accelerated conditions. A stable norepinephrine formulation also may refer to a formulation that contains from about 1% to about 10%, e.g., from about 1% to about 8%, from about 2% to about 6%, from about 0.5% to about 5%, from about 1.5% to about 4%, from about 1.0% to about 2.5%, or from about 0.5% to about 2%, of total norepinephrine-related impurities present in the formulation after storage under room temperature and/or accelerated conditions.

A stable norepinephrine formulation also may refer to a formulation that contains less than about 5% (area percent), e.g., less than about 4%, less than about 3%, less than about 2%, less than about 1%, less than about 0.8%, less than about 0.4%, or less than about 0.2%, of any individual norepinephrine-related impurity present in the formulation after storage under room temperature and/or accelerated conditions. A stable norepinephrine formulation additionally may refer to a formulation that contains about 0.1% to about 5%, about 0.2% to about 4%, about 0.4% to about 3%, about 0.4% to about 1.5%, about 0.5% to about 2%, or about 0.3% to about 3% of any individual norepinephrine-related impurity present in the formulation after storage under room temperature and/or accelerated conditions.

In some embodiments, the norepinephrine formulation of the invention is stable for at least about 9 months, e.g., at least about 12 months, at least about 18 months, at least about 24 months, or at least about 36 months at room temperature (e.g., at 25±2° C./60% RH±5% RH) or at refrigerated temperature (e.g., at 5±3° C.). The invention also includes embodiments in which the norepinephrine formulation of the invention is stable for at least about 1 month, e.g., at least about 3 months, at least about 6 months, or at least about 12 months under accelerated conditions (e.g., at 40° C.±2° C./75% RH±5% RH).

Methods for determining the stability of a formulation of the invention with respect to a given parameter are well-known in the art. For example, individual impurities and total impurities may be assessed by high-performance liquid chromatography (HPLC) or thin layer chromatography (TLC). Unless indicated otherwise, a percentage amount norepinephrine, any individual impurity, or total impurities reported herein in the formulation is determined by a peak area percent method using HPLC.

In some embodiments, a stable norepinephrine formulation may refer to a formulation that is colorless after storage under room temperature and/or accelerated conditions. The color of the formulation may be determined, for example, by a United States Pharmacopoeia (USP) or European Pharmacopoeia (Ph. Eur.) color method. For example, a stable norepinephrine formulation of the invention may refer to a formulation that has a coloration of not less than B8 as determined by Ph. Eur. Method 2.2.2 after storage for at least 6 months at room temperature. By way of further example, a stable norepinephrine formulation of the invention may refer to a formulation that has a coloration of not less than B8 as determined by Ph. Eur. Method 2.2.2 after storage for at least 1 month under accelerated conditions (e.g., at 40° C.±2° C./75% RH±5% RH).

The formulation may include a therapeutically effective amount of norepinephrine or a pharmaceutically acceptable salt thereof, such as, e.g., norepinephrine bitartrate, norepinephrine tartrate, or norepinephrine HCl. Preferably, the norepinephrine is of high enantiomeric purity or enantiomerically pure, e.g., at least about 95% R-isomer relative to all possible (R and S) enantiomers combined, at least about 97% R-isomer relative to all possible enantiomers, or at least about 99% R-isomer relative to all possible enantiomers. In some embodiments, the formulation includes a therapeutically effective amount of norepinephrine bitartrate, which is preferably of high enantiomeric purity or enantiomerically pure. The norepinephrine or pharmaceutically acceptable salt thereof in the formulation of the invention may be at a concentration of from about 0.5 μg/mL to about 200 μg/mL, e.g., from about 1 μg/mL to about 100 μg/mL, from about 5 μg/mL to about 50 μg/mL, from about 10 μg/mL to about 40 μg/mL, or from about 30 μg/mL to about 70 μg/mL. In some embodiments, the formulation includes about 16 μg/mL norepinephrine as free base. In other embodiments, the formulation includes about 32 μg/mL norepinephrine as free base.

The formulation may be provided in any suitable volume. In some embodiments, the volume of the formulation is about 5 mL or more, e.g., about 10 mL or more, about 50 mL or more, about 100 mL or more, about 150 mL or more, about 200 mL or more, or about 250 mL or more. In other embodiments, the volume of the formulation is about 1 L or less, e.g., about 750 mL or less, about 500 mL or less, about 400 mL or less, about 300 mL or less, about 250 mL or less, or about 200 mL or less. The formulation also may be provided in a volume bounded by any two of the aforementioned endpoints. For example, the formulation may be provided in a volume of from about 10 mL to about 200 mL, from about 50 mL to about 500 mL, from about 100 mL to about 400 mL, from about 150 mL to about 300 mL, or from about 200 mL to about 300 mL. In certain embodiments, the volume of the formulation is about 250 mL. One of ordinary skill in the art may readily select an appropriate container based upon the volume of the formulation.

The formulation of the invention may include at least one antioxidant. In some embodiments, the antioxidant may include an amino acid or pharmaceutically acceptable salt thereof. The amino acid may include an L-stereoisomer, a D-stereoisomer, or a combination thereof. In some embodiments, the amino acid is naturally occurring. For example, the amino acid may include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine, pyrrolysine, or a combination thereof. In some embodiments, the amino acid may include tryptophan, methionine, histidine, lysine, arginine, or tyrosine. The invention also includes embodiments in which the amino acid comprises at least two primary or secondary amine groups, such as arginine, asparagine, lysine, methyl lysine, or ornithine. In certain embodiments, the antioxidant includes arginine.

The antioxidant may be present in the formulation in any suitable concentration. For example, the antioxidant may be present in the formulation at a concentration of about 10 μg/mL or more, e.g., about 25 μg/mL or more, about 50 μg/mL or more, about 100 μg/mL or more, about 250 μg/mL or more, about 400 μg/mL or more, about 500 μg/mL or more, about 1 mg/mL or more, about 5 mg/mL or more, about 7.5 mg/mL or more, or about 10 mg/mL or more. Alternatively, the antioxidant may be present in the formulation at a concentration of about 50 mg/mL or less, for example, about 25 mg/mL or less, about 10 mg/mL or less, about 7.5 mg/mL or less, about 5 mg/mL or less, about 2.5 mg/mL or less, about 2 mg/mL or less, about 1 mg/mL or less, about 500 μg/mL or less, or about 100 μg/mL or less. The antioxidant also may be present in the formulation in a concentration bounded by any two of the aforementioned endpoints. For example, the antioxidant can be present in the formulation in a concentration of from about 25 μg/mL to about 25 mg/mL, e.g., from about 50 μg/mL to about 5 mg/mL, from about 50 μg/mL to about 500 μg/mL, from about 100 μg/mL to about 10 mg/mL, from about 250 μg/mL to about 5 mg/mL, from about 400 μg/mL to about 7.5 mg/mL, from about 500 μg/mL to about 2.5 mg/mL, from about 1 mg/mL to about 2.5 mg/mL, or from about 1 mg/mL to about 2 mg/mL. In some embodiments, the antioxidant includes arginine present at a concentration of about 1.7 mg/mL.

The present invention is based, at least in part, on the surprising and unexpected discovery that certain metal ion chelators and/or antioxidants that are commonly used in liquid pharmaceutical formulations intended for parenteral administration are not necessary to stabilize a liquid norepinephrine formulation. The invention accordingly includes embodiments in which the formulation is substantially free of a metal ion chelator and/or antioxidant, thereby advantageously avoiding the need to include and administer such additives by injection. The invention accordingly includes embodiments in which the formulation is substantially free of, for example, a sulfite or a bisulfite. The invention also includes embodiments in which the formulation is substantially free of, e.g., an aminopolycarboxylic acid such as, for example, ethylenediaminetetraacetic acid (EDTA), ethylene glycol-bis(3-aminoethyl ether)-N,N,N′,N′-tetraacetic acid (EGTA), or a salt thereof. The invention further includes embodiments in which the formulation is substantially free of, e.g., butylated hydroxyl anisole (BHA), butylated hydroxyl toluene (BHT), ascorbic acid, alpha-tocopherol, or propyl gallate. The term “substantially free” as used herein with respect to an excipient means that no amount of the excipient is added during manufacture of the formulation and, if present, only may be present as an impurity.

The invention also includes embodiments wherein an amino acid or pharmaceutically acceptable salt thereof and another antioxidant are included in the formulation. In some embodiments, the formulation comprises arginine, asparagine, lysine, methyl lysine, or ornithine, and a sulfite, bisulfite, BHA, BHT, ascorbic acid, alpha-tocopherol, or propyl gallate. In certain embodiments, the formulation comprises arginine and a sulfite, such as, for example, sodium metabisulfite. The amino acid and additional antioxidant may be present in the formulation in any suitable concentration as described herein with respect to antioxidants. In some embodiments, the formulation comprises from about 100 μg/mL to about 10 mg/mL of an amino acid, and from about 0.5 μg/mL to about 200 μg/mL of another antioxidant. In certain embodiments, the formulation comprises from about 0.5 mg/mL to about 5 mg/mL of arginine, and from about 1 μg/mL to about 50 μg/mL of sodium metabisulfite.

The formulation of the invention also may include at least one tonicity agent. Suitable tonicity agents may include, without limitation, sodium chloride, dextrose, mannitol, trehalose, potassium chloride, glycerol, or a combination thereof. In some embodiments, the tonicity agent includes sodium chloride. In other embodiments, the tonicity agent includes dextrose. In certain embodiments, the tonicity agent includes dextrose and the formulation is substantially free of sodium chloride. The tonicity agent is preferably present in an amount that renders the formulation isotonic. For example, the tonicity agent may present in an amount sufficient to provide the formulation with an osmolality of about 250-350 mOsm/kg, e.g., about 270-330 mOsm/kg, about 260-320 mOsm/kg, about 300-340 mOsm/kg, or about 310-330 mOsm/kg. In some embodiments, the tonicity agent is present in an amount that provides the formulation with an osmolality of 290 mOsm/kg±10%. The invention also includes embodiments in which the formulation includes about 50 mg/mL dextrose.

The formulation additionally may include at least one buffer. In some embodiments, however, the formulation is free of a buffer, or substantially free of a buffer. If present, the type and amount of buffer present in the formulation may be selected based on several considerations, including but not limited to, for example, a target pH, pH stabilization, impurity formation, coloration, and/or patient tolerance upon administration. In some embodiments, the buffer may include a weak acid and a conjugate base of the weak acid. The weak acid and conjugate base may be added to the formulation in an anhydrous or hydrated form. In some embodiments, the conjugate base may be present in salt form. The invention also includes embodiments in which the acid or weak acid component may be a dicarboxylic acid or a tricarboxylic acid. For example, the acid includes citric acid, isocitric acid, aconitic acid, trimesic acid, propane-1,2,3-tricarboxylic acid, fumaric acid, oxalic acid, maleic acid, malonic acid, glutaric acid, succinic acid, tartaric acid, or a combination thereof. In some embodiments, the buffer includes citric acid and a salt thereof (i.e., a citrate salt).

In other embodiments, the formulation is free of a dicarboxylic acid, a tricarboxylic acid, and salts thereof, or substantially free of a dicarboxylic acid, a tricarboxylic acid, and salts thereof. The invention accordingly includes embodiments in which the formulation is free of citric acid and a citrate salt, or substantially free of citric acid and a citrate salt.

One of ordinary skill in the art may readily determine the amount of buffer required to achieve or maintain the desired pH, for example, based upon a weak acid and conjugate base included in the formulation. In some embodiments, the buffer may be present at a concentration of about 50 mM or less, e.g., about 40 mM or less, about 30 mM or less, about 20 mM or less, about 10 mM or less, or about 5 mM or less. In some embodiments, the buffer may be present at a concentration of about 0.5 mM or more, e.g., about 1 mM or more, about 2 mM or more, about 5 mM or more, about 10 mM, or about 20 mM or more. The invention also includes embodiments in which the buffer is present at a concentration of about 0.5-40 mM, e.g., about 1-20 mM, about 2-12 mM, about 7-11 mM, or about 8-10 mM. In some embodiments, the buffer includes a citrate buffer present at a concentration of about 5-15 mM, and preferably at a concentration of about 7-11 mM, for example, about 9 mM of a citrate buffer.

The formulation of the invention may further include a pH adjuster. The pH adjuster may include any suitable pH adjuster. A suitable pH adjuster may include, for example, sodium hydroxide, potassium hydroxide, hydrochloric acid, or a combination thereof. In some embodiments, the pH adjuster includes sodium hydroxide, hydrochloric acid, or a combination thereof.

The formulation of the invention may have any suitable pH. In some embodiments, the formulation may have a pH of about 3 or more, e.g., about 3.1 or more, about 3.2 or more, about 3.3 or more, about 3.4 or more, about 3.5 or more, about 3.6 or more, about 3.7 or more, about 3.8 or more, about 3.9 or more, or about 4.0 or more. In some embodiments, the formulation may have a pH of about 4.5 or less, e.g., about 4.4 or less, about 4.3 or less, about 4.2 or less, about 4.1 or less, about 4.0 or less, about 3.9 or less, about 3.8 or less, about 3.7 or less, about 3.6 or less, or about 3.5 or less. The invention also includes embodiments in which the formulation may have a pH bounded by any two of the foregoing endpoints for the formulation. For example, the formulation may have a pH of from about 3.0 to about 4.5, from about 3.0 to about 4.0, from about 3.0 to about 3.8, from about 3.1 to about 3.7, from about 3.2 to about 3.6, from about 3.3 to about 3.7, or from about 3.4 to about 3.6. In some embodiments, the formulation has a pH of about 3.5.

The formulation of the invention may be formulated to exhibit a stable pH following storage under room temperature and/or accelerated conditions. In some embodiments, the pH drift of the formulation is less than about 0.4 pH units, e.g., less than about 0.3 pH units, less than about 0.25 pH units, less than about 0.2 pH units, less than about 0.15 pH units, less than about 0.1 pH units, or less than about 0.05 pH units, following storage for at least about 6 months at room temperature. The invention also includes embodiments in which the pH drift of the formulation is less than about 0.4 pH units, e.g., less than about 0.3 pH units, less than about 0.25 pH units, less than about 0.2 pH units, less than about 0.15 pH units, less than about 0.1 pH units, or less than about 0.05 pH units, following storage for at least about 12 months at room temperature. In some embodiments, the pH drift of the formulation is less than about 0.4 pH units, e.g., less than about 0.3 pH units, less than about 0.25 pH units, less than about 0.2 pH units, less than about 0.15 pH units, less than about 0.1 pH units, or less than about 0.05 pH units, following storage for at least about 24 months at room temperature.

In some embodiments, the invention provides a ready-to-administer, liquid formulation which includes a therapeutically effective amount of norepinephrine or a pharmaceutically acceptable salt thereof, an antioxidant, a tonicity agent, and water, wherein the formulation has a pH of from about 3.0 to about 3.8, and wherein the formulation is substantially free of a sulfite. The tonicity agent may include dextrose, and the formulation also may be substantially free of sodium chloride. The formulation further may be substantially free of an aminopolycarboxylic acid.

The invention also provides a ready-to-administer, liquid formulation which includes a therapeutically effective amount of norepinephrine or a pharmaceutically acceptable salt thereof, an amino acid, a tonicity agent, and water, wherein the formulation has a pH of from about 3.0 to about 3.8, and wherein the formulation is substantially free of a sulfite. The tonicity agent may include dextrose, and the formulation also may be substantially free of sodium chloride. The amino acid may include at least two primary or secondary amine or amino groups. For example, the amino acid may include arginine, asparagine, lysine, methyl lysine, ornithine, or a combination thereof. The formulation also may be substantially free of an aminopolycarboxylic acid.

The invention additionally provides a ready-to-administer, liquid formulation consisting essentially of a therapeutically effective amount of norepinephrine or a pharmaceutically acceptable salt thereof, an amino acid, a tonicity agent, and water, wherein the formulation has a pH of from about 3.0 to about 3.8. The tonicity agent may include dextrose. The amino acid may include at least two primary or secondary amine groups. For instance, the amino acid may include arginine, asparagine, lysine, methyl lysine, or ornithine, or a combination of two of more of such amino acids.

The invention further provides a ready-to-administer, liquid formulation which includes a therapeutically effective amount of norepinephrine or a pharmaceutically acceptable salt thereof, arginine, a tonicity agent, and water, wherein the formulation has a pH of from about 3.0 to about 3.8, and wherein the formulation is substantially free of a sulfite. The tonicity agent may include about 50 mg/mL dextrose, and the formulation also may be substantially free of sodium chloride. The formulation additionally may be substantially free of an aminopolycarboxylic acid.

The invention moreover provides a ready-to-administer, liquid formulation consisting essentially of a therapeutically effective amount of norepinephrine or a pharmaceutically acceptable salt thereof, arginine, a tonicity agent, and water, wherein the formulation has a pH of from about 3.0 to about 3.8. The tonicity agent may include about 50 mg/mL dextrose, and the formulation also may be substantially free of sodium chloride.

The invention furthermore provides a ready-to-administer, liquid formulation that includes from about 5 μg/mL to about 50 μg/mL norepinephrine base, from about 400 μg/mL to about 7.5 mg/mL arginine, a tonicity agent, and water, wherein the formulation has a pH of from about 3.0 to about 3.8, and wherein the formulation is substantially free of a sulfite. The tonicity agent may include about 50 mg/mL dextrose, and the formulation also may be substantially free of sodium chloride. The formulation further may be substantially free of an aminopolycarboxylic acid.

The invention also provides a ready-to-administer, liquid formulation consisting essentially of from about 5 μg/mL to about 50 μg/mL norepinephrine base, from about 400 μg/mL to about 7.5 mg/mL arginine, a tonicity agent, and water, wherein the formulation has a pH of from about 3.0 to about 3.8. The tonicity agent may include about 50 mg/mL dextrose. In some embodiments, the formulation has an osmolality of about 260-320 mOsm/kg.

The invention further provides a pharmaceutical product which includes a primary container with the ready-to-administer, liquid norepinephrine formulation of the invention contained therein, and a sealed secondary container which houses the primary container. The liquid formulation component of the pharmaceutical product may include formulations having the same composition and characteristics (e.g., stability) as described herein with respect to the formulation of the invention. The primary container may include, for example, a syringe, a cartridge, a vial, an ampoule, a bag, or a bottle. In some embodiments, the primary container includes a bag or a bottle.

The primary container may include, for example, a flexible, multi-layered bag. The bag may include a material which is chemically inert to the formulation, sterilizable, and weldable. Such materials include, without limitation, polyolefin polymers (e.g., a polyethylene or polypropylene), cycloolefin polymers or cycloolefin copolymers, polycarbonates, styrene polymers, and block co-polymers thereof. In some embodiments, a polyolefin may be combined with an elastomeric polymer, such as, e.g., a styrene-ethylene/butylene-styrene-triblock polymer (SEBS), a styrene-ethylene/propylene-styrene-triblock polymer (SEPS), a styrene-butadiene-styrene-triblock polymer (SBS), and/or a styrene-isoprene-styrene triblock polymer (SIS). In certain embodiments, the innermost layer of the multi-layered bag comprises polypropylene and SEBS. In other embodiments, the innermost layer of the multi-layered bag comprises a polymer of cyclic olefin such as cycloolefin homopolymer or cycloolefin copolymer or mixture thereof. In yet other embodiments, the innermost layer of the multi-layered bag comprises ethylene-vinyl acetate copolymer. Suitable flexible bags are described in U.S. Pat. Nos. 5,783,269, 7,875,016, 8,162,915, 7,828,787, and/or 8,118,802, which are incorporated herein by reference in their entireties, and marketed under the tradename, FREEFLEX™. Other flexible polymeric containers suitable for use with a formulation according to the invention include, without limitation, GALAXY™, VIAFLO™, INTRAVIA™, and EXCEL™ containers.

In some embodiments, the primary container is disposed within and enclosed by a secondary container, such as a blister package or an overwrap. The secondary container may include an overwrap with, e.g., a first foil, a second foil, and a seal disposed along a common peripheral edge of the first and second foils. The first and second foils of the secondary container overwrap may include multilayer films. In some embodiments, the secondary container is fully transparent to enable visual inspection of the primary container, labeling, and any other contents within the secondary container (e.g., oxygen absorber). The invention also includes embodiments in which the secondary container is fully intransparent, for example, an aluminum overpouch. The invention additionally includes embodiments in which the secondary container includes a completely or partially intransparent first foil and a completely or partially transparent second foil. Examples of secondary containers suitable for use in the present invention are described in US Patent Application Publication Nos. 2006/0240204 and 2019/0151202, which are incorporated herein by reference in their entireties.

The pharmaceutical product may further include an oxygen absorber that absorbs and removes or decreases the level of oxygen that may be present in the liquid norepinephrine formulation, in the headspace of the primary container, and/or within the secondary container after initial packaging, as well as oxygen that may permeate through the secondary container during the shelf life of the pharmaceutical product. The oxygen absorber may be provided in any suitable size, form, or shape including, for example, a sachet, pouch, capsule, label, strip, patch, canister, cartridge, lining, or sticker, etc. The oxygen absorber may be placed inside of the secondary container or adhered or integrated into the primary container and/or the secondary container. In some embodiments, the oxygen absorber may be in the form of a sachet or in the form of a canister. The pharmaceutical product of the invention also includes embodiments in which the oxygen absorber may be in the form of a label or in the form of a strip. The pharmaceutical product of the invention additionally includes embodiments in which the oxygen absorber may be in the form of a sticker or label that adheres to the secondary container or to the primary container. The pharmaceutical product of the invention further includes embodiments in which the oxygen absorber may be incorporated as part of the secondary container itself such as, for example, as part of a lid, film, or seal of the secondary container.

Suitable materials for oxygen absorbers may include, for example, metal-based substances that remove oxygen by reacting with it by chemical bonding, generally forming a metal oxide component. Metal-based substances may include, e.g., elemental iron as well as iron oxide, iron hydroxide, iron carbide, and the like, and combinations thereof. Other metals for use as oxygen absorbers may include, e.g., nickel, tin, copper, zinc, and combinations thereof. Metal-based oxygen absorbers may be provided in the form of a powder, e.g., to increase active surface area. Powder forms of suitable metal-based oxygen absorbers may be obtained by any known method including, but not limited to, atomization, milling, pulverization, and electrolysis. Additional materials for oxygen absorbers may include, e.g., low molecular weight organic compounds such as, e.g., ascorbic acid, sodium ascorbate, catechol and phenol, activated carbon, polymeric materials incorporating a resin and a catalyst, and combinations thereof. In some embodiments, the oxygen absorber includes a metal-based oxygen absorber, such as an iron-based oxygen absorber.

A formulation of the invention that includes a pharmaceutically effective amount of norepinephrine or a pharmaceutically acceptable salt thereof, an antioxidant, and a tonicity agent may be prepared by any suitable technique, many of which are known in the art. The formulation also may be prepared, e.g., in a batch or continuous process. In some embodiments, the formulation may be prepared by combining the components thereof in any order. The term “component” as used herein includes individual ingredients (e.g., norepinephrine bitartrate, antioxidant, tonicity agent, pH adjuster, optional buffer, etc.) as well as any combination of two or more individual ingredients. In some embodiments, the formulation may be formed by combining the components together in a vessel. Such components may be combined in any order.

Thus, the invention provides a method for making a ready-to-administer, liquid norepinephrine formulation that is stable for at least 9 months at room temperature. In some embodiments, the method includes (1) dissolving a tonicity agent and an antioxidant in water to form a first solution, (2) adjusting the first solution to pH of from about 3.0 to about 3.8 to form a second solution, (3) dissolving a pharmaceutically effective amount of norepinephrine or a pharmaceutically acceptable salt thereof in the second solution to form a norepinephrine solution, and (4) sterilizing the norepinephrine solution to provide the ready-to-administer, liquid norepinephrine formulation.

In some embodiments, the method of the invention includes adding water to a suitable vessel, adding the antioxidant and tonicity agent, either sequentially or together, and stirring the mixture until dissolution is complete. Next, the pH may be adjusted to the desired value by adding one or more pH adjusters. Subsequently, the norepinephrine or pharmaceutically acceptable salt thereof may be added, and the mixture stirred until dissolution is complete or substantially complete. Next, the volume of the formulation may adjusted to a desired volume with water, filtered through one or more sterilizing filters, and filled into primary containers. Then, the primary container may be sealed and placed into a secondary container, which may then be sealed. In some embodiments, an oxygen absorber may be placed into the secondary container before it is sealed. Preferably, dissolved oxygen is removed, e.g., by nitrogen sparging, at one or more steps of the compounding, filling, and/or packaging processes.

In some embodiments, the sealed, pharmaceutical product is sterilized by terminal sterilization, e.g., autoclaving. The pharmaceutical product also may be manufactured using aseptic processing techniques, such that terminal sterilization is not required.

The invention also provides a method of stabilizing a norepinephrine formulation by forming a mixture which includes norepinephrine or a pharmaceutically acceptable salt thereof, an antioxidant, a tonicity agent, and water, thereby stabilizing the formulation. The type/form and amounts of norepinephrine or pharmaceutically acceptable salt thereof, antioxidant, and tonicity agent present in the mixture, as well as the pH, may include the same types/forms and amounts of these components and the pH described herein with respect to a formulation of the invention. The formulation produced by the inventive method of stabilizing a norepinephrine formulation may have the same stability characteristics as the stability characteristics described herein with respect to a formulation of the invention, particularly with regard to API assay and total impurities.

The formulation according to the invention is suitable for administration to a subject to treat or prevent a disease or condition, including a disease or condition that is treatable with norepinephrine or a pharmaceutically acceptable salt thereof. Preferably, the subject is a mammal such as, for example, a human. The disease or condition that is treatable by the administration of norepinephrine or a pharmaceutically acceptable salt thereof may include, for example, low blood pressure. In some embodiments, the condition may include severe, acute hypotension.

The following examples further illustrate the invention but, of course, should not be construed as in any way limiting its scope.

### Example 1

This example demonstrates the stability of exemplary formulations comprising norepinephrine, a tonicity agent, and an antioxidant.

Samples containing 32 μg/mL norepinephrine (as bitartrate), 7 mg/mL sodium chloride, 0.242 mg/mL citric acid (anhydrous) and 2.255 mg/mL trisodium citrate dihydrate, an antioxidant selected from 0.2 mg/mL sodium metabisulfite, 1 mg/mL cysteine, 2 mg/mL glutathione, or 1.7 mg/mL arginine, and water were adjusted to pH 3.5, filled into IV bags, placed into aluminum overwraps containing an oxygen absorber, and sealed using aseptic technique. The samples were placed into stability chambers under room temperature (25° C.±2° C./60% RH±5% RH) or accelerated temperature (e.g., at 40° C.±2° C./75% RH±5% RH) storage conditions for 1-3 months, and then analyzed by HPLC for norepinephrine content and total impurities.

The HPLC conditions were as follows:
Column: ACE 3C18 PFP 150×4.6 mm, PN EXL-1110-15464
Mobile Phase: 20 mm ammonium formate, pH 3.0 (11.34 g ammonium formate in 9 L water,
pH adjusted to 3.0 with H₃PO₄)
Column temperature: 25° C.
Flow rate: 1.0 mL/min
Injection volume: 30 μL
Autosampler temperature: 5° C.
Detection UV: 260 nm
Separation mode: Isocratic
Run time: 60 minutes

The results for norepinephrine content (API assay) and total impurities as determined by peak area percent are summarized in Table 1.

TABLE 1

| | | 25° C. | | 40° C. | |
|---|---|---|---|---|---|
| | Storage | Assay | Total | Assay | Total |
| Antioxidant | duration | % | Imp. | % | Imp. |
| Sodium | 1 month | 100.6 | 0.00 | 98.6 | 1.2 |
| Metabisulfite | 2 months | — | — | 94.2 | 3.29 |
| | 3 months | 100.5 | 0.00 | 94.3 | 4.5 |
| Cysteine | 1 month | 101 | 0.4 | 99.2 | 4.3 |
| | 2 months | — | — | 95.4 | 4.97 |
| Glutathione | 1 month | 99.9 | 13.3 | 99.2 | 6.8 |
| Arginine | 1 month | 100.6 | 0.0 | 99.4 | 2.50 |
| | 2 months | — | — | 99.9 | 1.88 |
| | 3 months | 100.5 | 0.0 | 97.6 | 2.60 |

The results described in this example demonstrate that the exemplary norepinephrine formulations comprising arginine had higher API assay and/or lower total impurities as compared with norepinephrine formulations comprising sodium metabisulfite, cysteine, or glutathione.

### Example 2

This example demonstrates the stability of exemplary formulations comprising norepinephrine, a tonicity agent, and an antioxidant.

Samples containing 16 μg/mL or 32 μg/mL norepinephrine (as bitartrate), 1.7 mg/mL arginine, a tonicity agent selected from 7 mg/mL sodium chloride or 50 mg/mL dextrose, and water were adjusted to pH 3.5, filled into IV bags, placed into aluminum overwraps containing an oxygen absorber, and sealed using aseptic technique. The samples containing sodium chloride additionally contained 0.242 mg/mL citric acid (anhydrous) and 2.255 mg/mL trisodium citrate dihydrate. The samples were placed into stability chambers under room temperature (25° C.±2° C./60% RH±5% RH) or accelerated temperature (e.g., at 40° C.±2° C./75% RH±5% RH) storage conditions for 0-9 months, and then analyzed by HPLC as described in Example 1.

The results for norepinephrine content (API assay), individual impurities eluting at relative response time (RRT) 12.4, 14.4, and 15.1, and total impurities as determined by peak area percent are summarized in Table 2.

TABLE 2

| | 25° C | | | | 40° C | | | | | |
|---|---|---|---|---|---|---|---|---|---|---|
| | | Assay | RRT | Total | | Assay | RRT | RRT | RRT | Total |
| Sample | time | % | 12.4 | Imp. | time | % | 12.4 | 14.4 | 15.1 | Imp. |
| 32 μg Nor. | 1 M | 96.0 | 0.63 | 0.63 | 1 M | 95.0 | 0.80 | 0.00 | 0.35 | 1.49 |
| (NaCl/citrate) | 9 M | 101.6 | 1.07 | 1.54 | 3 M | 100.2 | 1.63 | 0.55 | 0.76 | 3.21 |
| 32 μg Nor. | 1 M | 96.8 | 1.03 | 1.03 | 1 M | 96.0 | 2.88 | 0.42 | 0.00 | 3.44 |
| (Dextrose) | 9 M | 101.9 | 1.51 | 1.51 | 3 M | 103.5 | 1.61 | 0.00 | 0.00 | 2.89 |
| 16 μg Nor. | T0 | 101.1 | 0.00 | 0.00 | T0 | 101.1 | 0.00 | 0.00 | 0.00 | 0.00 |
| (NaCl/citrate) | 9 M | 102.7 | 2.66 | 3.74 | 3 M | 102.1 | 3.57 | 0.62 | 0.91 | 5.55 |
| 16 μg Nor. | T0 | 100.4 | 0.00 | 0.00 | T0 | 100.4 | 0.00 | 0.00 | 0.00 | 0.00 |
| (Dextrose) | 9 M | 101.7 | 2.70 | 2.70 | 3 M | 101.2 | 3.33 | 0.00 | 0.00 | 4.10 |

The results of this example demonstrate that the exemplary norepinephrine formulations comprising arginine and dextrose had lower individual impurities (particularly RRT 14.4 and 15.1) and/or lower total impurities as compared with norepinephrine formulations comprising arginine, sodium chloride, and citrate buffer, particularly in formulations comprising 16 μg/mL norepinephrine.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A ready-to-administer liquid formulation comprising:
a therapeutically effective amount of norepinephrine or a pharmaceutically acceptable salt thereof;
arginine;
a tonicity agent; and
water;
wherein the formulation has a pH of from about 3.0 to about 4.5 and is stable for at least 9 months at room temperature.

2. The formulation of claim 1, wherein the norepinephrine or pharmaceutically acceptable salt thereof comprises norepinephrine bitartrate at a concentration of from about 1 μg/mL to about 100 μg/mL.

3. The formulation of claim 1, wherein the formulation is substantially free of an aminopolycarboxylic acid and/or a sulfite.

4. The formulation of claim 1, wherein the arginine is present at a concentration of from about 400 μg/mL to about 7.5 mg/mL.

5. The formulation of claim 1, wherein the formulation has a pH of from about 3.6 to about 3.8.

6. The formulation of claim 1, wherein the tonicity agent comprises sodium chloride.

7. The formulation of claim 1, wherein the formulation comprises about 5-15 mM of a citrate buffer.

8. The formulation of claim 1, wherein the formulation has a pH drift of less than about 0.3 pH units following storage for at least about 6 months at room temperature.

9. The formulation of claim 1, wherein the formulation contains not more than not more than about 3% total impurities as determined by a peak area percent method by high-performance liquid chromatography (HPLC) after storage for at least about 3 months at 40° C.±2° C./75% RH±5% RH.

10. A pharmaceutical product comprising a primary container comprising the ready-to-administer formulation according to claim 1, and a sealed secondary container which houses the primary container.

11. The pharmaceutical product according to claim 10, wherein the primary container comprises a flexible, multi-layer bag comprising a polyolefin.

12. The pharmaceutical product according to claim 10, wherein the secondary container comprises an aluminum overwrap.

13. The pharmaceutical product according to claim 10, further comprising an oxygen absorber.

14. A ready-to-administer, liquid formulation consisting essentially of from about 5 μg/mL to about 50 μg/mL norepinephrine base, from about 400 μg/mL to about 7.5 mg/mL arginine, a tonicity agent, and water, wherein the formulation has a pH of from about 3.0 to about 4.5 and is stable for at least 9 months at room temperature.

15. The formulation of claim 14, wherein the formulation has a pH of from about 3.6 to about 3.8.

16. The formulation of claim 14, wherein the tonicity agent comprises sodium chloride.

17. The formulation of claim 14, wherein the formulation comprises about 5-15 mM of a citrate buffer.

18. The formulation of claim 14, wherein the formulation has a pH drift of less than about 0.3 pH units following storage for at least about 6 months at room temperature.

19. The formulation of claim 14, wherein the formulation contains not more than not more than about 3% total impurities as determined by a peak area percent method by high-performance liquid chromatography (HPLC) after storage for at least about 3 months at 40° C.±2° C./75% RH±5% RH.

20. A pharmaceutical product comprising a primary container comprising the ready-to-administer formulation according to claim 14, and a sealed secondary container which houses the primary container.
